(19) United States
(12) Patent Application Publication
(10) Pub. No.: US 2023/0416763 A1
(43) Pub. Date: Dec. 28, 2023

(54) STORING AND/OR TRANSPORTING EXTRACELLULAR NUCLEIC ACIDS

(71) Applicant: ATELERIX LIMITED, Cambridgeshire (GB)

(72) Inventors: Dean HALLAM, Cambridgeshire (GB); Stephen SWIOKLO, Cambridgeshire (GB); Che CONNON, Cambridgeshire (GB)

(21) Appl. No.: 18/037,119

(22) PCT Filed: Nov. 12, 2021
(86) PCT No.: PCT/GB2021/052936

§ 371 Date: May 16, 2023

(30) Foreign Application Priority Data
Nov. 16, 2020 (GB) 2017996.6

(51) Int. Cl. C12N 15/68

(52) U.S. Cl. CPC C12N 15/68

(57) ABSTRACT

The present invention provides a reversibly cross-linked hydrogel comprising a sample, wherein the sample comprises extracellular nucleic acid. Corresponding methods of preparing, transporting and/or storing the nucleic acid-containing hydrogel, as well as uses thereof, are also provided herein.

The present invention provides a reversibly cross-linked hydrogel comprising a sample, wherein the sample comprises extracellular nucleic acid. Corresponding methods of preparing, transporting and/or storing the nucleic acid-containing hydrogel, as well as uses thereof, are also provided herein.

### BACKGROUND

Extracellular nucleic acids are playing an increasingly important role in society as highlighted by the recent COVID-19 vaccine trials and emerging COVID-19 diagnostic assays. They may be used in several contexts, including scientific research, drug development, regenerative medicine, diagnostic assays and vaccine development.

Nucleic acid based products such as nucleic acid based vaccines may be generated and/or prepared for use in a location that is often geographically separated from their point of use. Furthermore, nucleic acid samples (e.g. patient samples for viral testing) may be obtained in a different location to where diagnosis takes place. Samples comprising nucleic acids are therefore routinely stored and/or transported from a first geographical location to a second geographical location. However, shipping of such materials within the UK or globally can take hours or days and is vulnerable to delays, and the material needs to be delivered to the point of use in a condition that is fit for purpose. Effective transportation and recovery of nucleic acids (particularly RNA) has proven difficult, with many methods resulting in nucleic acid degradation and/or loss of function over time. Storage and/or transport of nucleic acids therefore represents a significant barrier in respect of e.g. laboratory supply (distribution for research) and diagnostics or therapeutics.

Conventional methods for the storage and shipment of nucleic acids include either cold-chain shipping (e.g. at 2-8° C.) or freezing the sample prior to, and during, shipping. However, such methods generally require that a number of processing steps are carried out prior to shipping, and these processes may adversely affect the shipped material or significantly increase cost. Conventional methods for the storage and shipment of nucleic acids therefore have a number of drawbacks. These drawbacks are a particularly significant problem when the extracellular nucleic acid is for human use (e.g. as a vaccine) or is for diagnostic purposes, where maintaining the nucleic acid structural integrity and/or functionality is key.

There is a need for a simple yet effective method for storing and/or transporting extracellular nucleic acid.

### BRIEF SUMMARY OF THE DISCLOSURE

The inventors have developed a novel means for storing and/or transporting extracellular nucleic acid.

The inventors have surprisingly shown that incorporation of extracellular nucleic acid into a reversibly cross-linked hydrogel protects the extracellular nucleic acid from the mechanical and environmental stresses of storage and preserves the structural integrity and/or functionality of the extracellular nucleic acid. Advantageously, a hydrogel comprising extracellular nucleic acid can be packaged in a sealed receptacle for effective storage or delivery to its point of use, whilst maintaining the material in a condition that is fit for purpose. Furthermore, storage and/or transportation of the packaged material can effectively be undertaken for longer periods of time without significantly impacting on the structural integrity and/or functionality of the nucleic acid.

The methods of the invention may be particularly useful for storing extracellular nucleic acid (such as isolated or manufactured extracellular nucleic acid, including viral vectors and viruses) immediately, before any deterioration has occurred and this provides flexibility to the user, as the extracellular nucleic acid (e.g. isolated/manufactured extracellular nucleic acid, including viral vectors and viruses) can be safely stored until the appropriate staff are available, a GMP laboratory is accessible or until samples can be processed in bulk, without impacting endpoint performance.

The invention has been exemplified using alginate hydrogels. However, the invention applies equally to other reversibly cross-linked hydrogels with the equivalent mechanical properties. Alternative hydrogels that may be equally used within the context of the invention are described in more detail below.

Furthermore, the invention has been exemplified using a virus, particularly human coronavirus 229E. The inventors have shown herein that the coronavirus 229E nucleic acid is preserved and maintains its functionality after storage in the hydrogels described herein. Surprisingly, even after seven to fourteen days of storage in the hydrogel, the stored coronaviruses were shown to maintain functionality (when viral cytopathic effect (CPE) was determined). This shows an improved preservation of nucleic acid functionality by the hydrogels provided herein compared to conventional means for storing such nucleic acids (e.g. compared to viral nucleic acid storage in Virocult®). The hydrogels may be used to preserve the functionality of any extracellular nucleic acid, including extracellular nucleic acids that are, for example, present within a viral particle, and/or extracellular nucleic acids that are present within a lipid capsule such as a lipid membrane.

In one aspect, a reversibly cross-linked hydrogel comprising a sample is provided, wherein the sample comprises extracellular nucleic acid.

Suitably, the extracellular nucleic acid may be within a lipid capsule.

Suitably, the extracellular nucleic acid may be in a viral vector.

Suitably, the extracellular nucleic acid may be in a viral particle.

Suitably, the sample may further comprise an aqueous buffer.

Suitably, the aqueous buffer may be a salt-based buffer.

Suitably, the hydrogel may comprise cross-linked alginate.

Suitably, the hydrogel may comprise cross-linked calcium-alginate, strontium-alginate, barium-alginate, magnesium-alginate and/or sodium-alginate.

Suitably, the cross-linked alginate may comprise from about 0.1% (w/v) to about 5.0% (w/v) calcium alginate.

Suitably, the extracellular nucleic acid may be RNA or DNA.

Suitably, the hydrogel may be packaged in a sealed receptacle.

Suitably, the sealed receptacle may be a vial, tube, flask, dish, vessel or plate.

In another aspect, a method of preparing a sample comprising extracellular nucleic acid for storage and/or transportation from a first location to a second location is provided, the method comprising the steps of:
i) contacting the sample with a hydrogel-forming polymer; and
ii) polymerising the polymer to form a reversibly cross-linked nucleic acid-containing hydrogel.

Suitably, the method may further comprise mixing the extracellular nucleic acid with an aqueous buffer prior to step (i). Optionally, the aqueous buffer may be a salt-based buffer.

Suitably, the method may comprise sealing the nucleic acid-containing hydrogel into a receptacle for storage or transportation from the first location to the second location. Optionally, the sealed receptacle may be a vial, tube, flask, dish, vessel or plate.

Suitably, the method may further comprise:
iii) dispatching the sealed receptacle for transportation from the first location to the second location.

In another aspect, a method of transporting a sample comprising an extracellular nucleic acid from a first location to a second location is provided, the method comprising the steps of:
(a) obtaining a reversibly cross-linked nucleic acid-containing hydrogel generated according to a method of the invention; or obtaining a reversibly cross-linked nucleic acid-containing hydrogel of the invention;
(b) transporting the hydrogel from the first location to the second location.

Suitably, the method may further comprise releasing the nucleic acid from the hydrogel at the second location.

In another aspect, a method of storing a sample comprising an extracellular nucleic acid is provided, the method comprising the steps of:
(a) obtaining a reversibly cross-linked nucleic acid-containing hydrogel generated according to a method of the invention; or obtaining a reversibly cross-linked nucleic acid-containing hydrogel of the invention; and
(b) storing the hydrogel.

Suitably, the method may further comprise releasing the nucleic acid from the hydrogel after storage.

In another aspect, a method for fulfilling an order or request for an extracellular nucleic acid is provided herein, the method comprising the steps of:
a) receiving an order or request for an extracellular nucleic acid;
b) obtaining a reversibly cross-linked nucleic acid-containing hydrogel generated according to a method of the invention; or obtaining a reversibly cross-linked nucleic acid-containing hydrogel of the invention; and
c) dispatching the sample for transportation; or transporting the sample to the location specified in the order or request.

Suitably, the extracellular nucleic acid may be within a lipid capsule.

Suitably, the extracellular nucleic acid may be in a viral vector.

Suitably, the extracellular nucleic acid may be in a viral particle.

Suitably, the sample may further comprise an aqueous buffer.

Suitably, the aqueous buffer may be a salt-based buffer.

Suitably, the hydrogel may comprise cross-linked alginate.

Suitably, the hydrogel may comprise cross-linked calcium-alginate, strontium-alginate, barium-alginate, magnesium-alginate and/or sodium-alginate.

Suitably, the cross-linked alginate may comprise from about 0.1% (w/v) to about 5.0% (w/v) calcium alginate.

Suitably, the extracellular nucleic acid may be RNA or DNA.

In another aspect, the use of a reversibly cross-linked hydrogel for preserving extracellular nucleic acid is provided.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows Log TCID50/mL for human coronavirus 229E (CoV 229E) following incubation in Atelerix SwabReady™ and Virocult® viral transport medium for up to 14 days. The limit of detection is 1.5 Log.

FIG. 2 shows the average recovery for Adenovirus (type V) following incubation in Atelerix SwabReady™ and Phosphate buffered saline (PBS) for up to 14 days. Black dashed line shows limit of detection (1.5 Log).

FIG. 3 shows the average recovery of Visna virus following incubation in Atelerix SwabReady™ for 3, 7 and 14 days when compared to the Phosphate buffered saline control at Day 0. Black dashed line shows limit of detection (1.5 Log). PBS=Phosphate buffered saline.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

A reversibly cross-linked hydrogel comprising a sample is provided herein, wherein the sample comprises extracellular nucleic acid.

As used herein “nucleic acid”, “nucleic acid sequence”, “oligonucleotide”, “polynucleotide”, “nucleic acid molecule” and variations thereof are used interchangeably to refer to plurality of nucleotides in either a regular or irregular sequence. Polynucleotides are typically single-stranded or double-stranded (duplex), but may adopt higher-order structures that contain three (triplex) or four (quadruple)di-motif) strands, or may contain a mix of these configurations in different loci under suitable conditions. Polynucleotides may be short or long. They have at least two adjacent nucleotides.

The nucleotide sequence may be of genomic, synthetic or recombinant origin, and may be double-stranded or single-stranded (representing the sense or antisense strand). The term “nucleic acid” includes genomic DNA, cDNA, synthetic DNA, RNA (e.g. mRNA), a chimeric DNA/RNA molecule and analogs of the DNA or RNA generated, e.g., by the use of nucleotide analogs. In other words, modified DNA or RNA bases are also encompassed. The polynucleotide may therefore include one or a plurality of modified DNA or RNA bases.

Several modified bases are known in the art and suitable modified bases can therefore be readily identified by a person of skill in the art. Polynucleotides bearing multiple modifications at specific sites have applications in synthetic biology, nanomaterial fabrication, bioanalytical, and sequencing applications. For example, DNA can be chemically modified at any, or all, of its three component parts—the phosphate linkage, the sugar ring, or the nucleobase. A variety of modified nucleotides can be obtained commercially as deoxynucleotidetriphosphates (dNTPs) or as phosphoramidite derivatives. These and other modified nucleotides can be synthesised and inserted into DNA or RNA either enzymatically as dNTPs, or through automated DNA synthesis as phosphoramidites. Methods for generating nucleic acids are well known in the art and include recombinant DNA techniques (i.e. recombinant DNA).

Nucleotide residues are usually derived from the naturally occurring purine bases, namely adenine (A), guanine (G), hypoxanthine (I), and xanthine (X), and pyrimidine bases, namely cytosine (C), thymine (T), and uracil (U). Nucleotide analogues may be used at one or more of the positions within the polynucleotide sequence, such nucleotide analogues being modified in e.g. the base portion and/or the sugar portion and/or the phosphate linkage. Any nucleotide analogue can be used provided that it does not prevent the polynucleotide from hybridising and that it is accepted by polymerase as both a template and a substrate.

Any suitable nucleic acid may be used herein. In one example, the nucleic acid is DNA. In another example, the nucleic acid is RNA (e.g. single stranded or double stranded RNA). DNA or RNA nucleic acids may be useful in vaccines, for example as part of a viral vector or a viral particle. In a particular example, the nucleic acid is RNA. In a specific example, the nucleic acid is single stranded RNA (e.g. a single stranded RNA virus).

In a particular example the nucleic acid is mRNA. mRNA may be particularly useful as the nucleic acid component of a vaccine (e.g. wherein the mRNA may be encapsulated in a lipid nanoparticle). In a particular example, therefore, a reversibly cross-linked hydrogel comprising a sample is provided, wherein the sample comprises extracellular mRNA.

As used herein “extracellular” refers to being situated or taking place outside a cell or cells. An extracellular nucleic acid is not located within a cell.

The term “extracellular nucleic acids” encompasses “cell-free” nucleic acids (i.e. nucleic acids that are not associated with a cell). As used herein, “associated with” refers to the physical location and/or interaction between two entities. In the context of nucleic acids and cells, a nucleic acid may “associated with” a cell when it is located within a cell, or when it is attached to the cell surface (i.e. physically interacting with the cell or within the cell). A cell-free nucleic acid is not associated with a cell and thus is not located within a cell and is not attached to a cell surface.

For the avoidance of doubt, the term “extracellular” or “cell-free” in this context refers to the physical state of the nucleic acid when it is introduced to the hydrogel (i.e. the physical state of the nucleic acid when it is directly or indirectly contacted with the hydrogel). In this context, “directly contacted” refers to situations where the nucleic acid is itself in physical contact with the hydrogel (e.g. when the nucleic acid is not within a lipid capsule), whereas “indirectly contacted” refers to situations where the nucleic acid itself is not in direct contact with the hydrogel, for example, when the nucleic acid is within a lipid capsule (described in more detail elsewhere herein).

The term “extracellular” (or “cell-free”) therefore encompasses nucleic acids that originate within a cell but are no longer located within a cell or associated with a cell (including the cell from which they originated). In other words, an extracellular nucleic acid (or cell-free nucleic acid) includes a nucleic acid that was generated within a cell, then released or isolated from the cell before contacting the nucleic acid with the hydrogel. In some examples, the extracellular nucleic acid may be an extraneous nucleic acid (in other words, a nucleic acid that is of external origin, that is, a nucleic acid that does not originate from a cell). For example, the nucleic acid may be a synthetic nucleic acid.

The extracellular nucleic acids described herein may be for any suitable application. Several suitable applications are well known to a person of skill in the art.

For example, the extracellular nucleic acid may be for use as a vaccine, e.g. a vaccine comprising the nucleic acid. Examples of such vaccines may be mRNA vaccines (e.g. where the mRNA is within a lipid nanoparticle), or viral vector vaccines (e.g. wherein the nucleic acid is within a viral vector) including virus-based vaccines (wherein the nucleic acid is within a synthetic or recombinant virus particle, or a naturally occurring virus particle such as an inactivated virion).

In another example, the extracellular nucleic acid may be for use in diagnostics. For example, the extracellular nucleic acid may be present in a sample that has been obtained from a subject (e.g. obtained from the subject's throat and/or nose using a swab). As a non-limiting example, the subject may be suspected to have, or suspected to be at risk of having, an infection, disease or disorder that may be identified by the presence of the extracellular nucleic acid in a sample obtained from the subject. For example, the subject may be suspected to have, or suspected to be at risk of having a viral infection, such as, but not limited to a COVID-19 infection. The presence of viral (e.g. COVID-19) nucleic acid within the sample may therefore be indicative of an infection. Preservation of the nucleic acid in the sample until diagnosis is possible is particularly important in such contexts.

As a further non-limiting example, the extracellular nucleic acid may be within a viral particle, wherein the presence of the viral particle may be useful for diagnosis. For example, the viral particle may be present in a sample that has been obtained from a subject (e.g. obtained from the subject's throat and/or nose using a swab). As a non-limiting example, the subject may be suspected to have, or suspected to be at risk of having, an infection, disease or disorder that may be identified by the presence of the viral particle in a sample obtained from the subject. For example, the subject may be suspected to have, or suspected to be at risk of having a viral infection, such as, but not limited to a COVID-19 infection. The presence of viral (e.g. COVID-19) nucleic acid or viral particle proteins (e.g. COVID-19 spike protein, N-protein or M-protein) within the sample may therefore be indicative of an infection. Preservation of the viral particle in the sample until diagnosis is possible is particularly important in such contexts.

The term “subject” as used herein refers to a mammal. A subject therefore refers to, for example, dogs, cats, horses, cows, pigs, guinea pigs, and the like. The subject can be a human. When the subject is a human, the subject may be referred to herein as a patient. The terms “subject”, “individual”, and “patient” are used herein interchangeably.

The subject can be symptomatic (e.g., the subject presents symptoms associated with an infection, disease or disorder, e.g. a viral infection such as COVID-19), or the subject can be asymptomatic (e.g., the subject does not present symptoms associated with an infection, disease or disorder, e.g. a viral infection such as COVID-19).

As described above, in one particular example, the nucleic acid may be in a vector.

A vector is a tool that allows or facilitates the transfer of an entity from one environment to another. By way of example, some vectors used in recombinant nucleic acid techniques allow entities, such as a segment of nucleic acid (e.g. a heterologous DNA segment, such as a heterologous cDNA segment), to be transferred into and expressed by a target cell. The vector may facilitate the integration of the nucleic acid/nucleotide of interest (NOI) to maintain the NOI and its expression within the target cell. Alternatively, the vector may facilitate the replication of the vector through expression of the NOI in a transient system. The vector may serve the purposes of maintaining the heterologous nucleic acid (DNA or RNA) within the cell, or facilitating the replication of the vector comprising a segment of DNA or RNA or the expression of the protein encoded by a segment of nucleic acid. The vector may facilitate the integration of the nucleic acid/nucleotide of interest (NOI) to maintain the NOI and its expression within the target cell. Alternatively, the vector may facilitate the replication of the vector through expression of the NOI in a transient system.

In a particular example, the nucleic acid may be within a vector such as a viral vector. Accordingly, in one example, a reversibly cross-linked hydrogel comprising a sample is provided, wherein the sample comprises extracellular nucleic acid in a viral vector.

The use of viral vectors for delivery of therapeutic genes is well known and gene therapy products are now an important part of our global healthcare markets. Such viral vectors may be particularly useful as viral vaccines. Examples of viral vectors include retroviral vectors (such as lentiviral vectors e.g. visna virus), adenoviral and adeno-associated viral vectors, herpes simplex virus vectors and vaccinia virus vectors. Specific examples within each of these groups of vectors are well known in the art (see for example Coffin et al. (1997) “Retroviruses”, Cold Spring Harbour Laboratory Press Eds: J M Coffin, SM Hughes, HE Varmus pp 758-763 for further details on retroviruses).

For example, a reversibly cross-linked hydrogel described herein may comprise a sample, wherein the sample comprises extracellular nucleic acid in an adenoviral vector.

The term “viral vector” encompasses a viral particle (as a viral particle also allows or facilitates the transfer of the nucleic acid from one environment to another). It encompasses natural, synthetic and recombinant viral particles.

The terms “viral particle”, “virus particle” and “virus” are used interchangeably herein. A viral particle comprises a nucleic acid (either DNA or RNA) encoding one or more viral components, surrounded by a protective protein coat called a capsid. The capsid may also be surrounded by an additional coat called the envelope, generating an “enveloped viral particle”. The envelope is typically derived from portions of the host cell membranes (phospholipids and proteins), with the addition of some viral glycoproteins. Enveloped viral particles therefore typically comprise a lipid capsule that protects the nucleic acid within the capsid.

Accordingly, in one example, a reversibly cross-linked hydrogel comprising a sample is provided, wherein the sample comprises extracellular nucleic acid in a viral particle. In a further example, a reversibly cross-linked hydrogel comprising a sample is provided, wherein the sample comprises extracellular nucleic acid in an enveloped viral particle.

The viral particle may be any viral particle including viruses that infect humans, including but not limited to human coronavirus (e.g. Human coronavirus 229E (CoV 229E)), human rhinovirus, and human adenovirus. It may also be e.g. a Visna virus.

Coronaviruses are enveloped, single stranded RNA viruses responsible for a variety of upper-respiratory tract illnesses in humans. These illnesses range from mild conditions such as the common cold to severe acute respiratory syndrome as seen in the recent COVID-19 pandemic. Coronaviruses are thought to be predominantly transmitted through respiratory droplets with some evidence to suggest the virus can remain active on fomites for several days. Interventions, both preventative and curative, are essential to slowing and/or stopping the spread of coronaviruses.

Human rhinoviruses are positive-sense single-stranded RNA viruses. They are the predominant cause of the common cold in humans. Rhinovirus infection proliferates in temperatures of 33-35° C., the temperatures found in the nose. Rhinoviruses belong to the genus Enterovirus in the family Picornaviridae.

Human adenoviruses are icosahedral viruses with double-stranded DNA. More than 50 distinct adenoviral serotypes have been found to cause a wide range of illnesses, from mild respiratory infections in young children (known as the common cold) to life-threatening multi-organ disease in people with a weakened immune system.

Other non-limiting examples of viruses that infect humans (which may be stored and/or transported in the hydrogels described herein) include viruses from the following families; Parvoviridae, Picornaviridae, Rhabdoviridae, Polyornaviridae, Reoviridae, Togaviridae, Bunyaviridae, Herpesviridae, Poxviridae, Flavivirid-ae, Orthomyxoviridae, Filoviridae, pararnyxoviridae, Hepadnaviridae, Adenoviridae, Astroviridae, Coronaviridae, Retroviridae, Papillornaviridae, Pneumoviridae, Arenaviridae, Caliciviridae, Anelloviridae etc.

The term “viral particle” encompasses synthetic or recombinant virus particles, or naturally occurring virus particles. It also encompasses non-infectious and infectious forms of the virus. Infectious forms are also referred to as virions. A reversibly cross-linked hydrogel comprising a sample, wherein the sample comprises extracellular nucleic acid in a virion (e.g. an enveloped virion) is therefore also provided herein.

Infectious virions may be inactivated to form an inactivated virion (e.g. for use as a vaccine). A reversibly cross-linked hydrogel comprising a sample, wherein the sample comprises extracellular nucleic acid in an inactivated virion (e.g. an inactivated SARs-CoV-2 virion) is therefore also provided herein.

As would be clear to a person of skill in the art, a sub-group of viral particles comprise an envelope, and thus may be considered to have a “lipid capsule”. In such cases, the nucleic acid is within a lipid capsule as defined in more detail elsewhere herein. For the avoidance of doubt, therefore, reference herein to extracellular nucleic acids that are “within a lipid capsule” also includes nucleic acids that are within enveloped viral particles, as well as extracellular nucleic acids that are within a different type of lipid capsule (e.g. liposomes, lipid nanoparticles, extracellular vesicles, micelles, lipid droplets, etc).

The extracellular nucleic acid described herein may be located within a lipid capsule. Accordingly, a reversibly cross-linked hydrogel described herein may comprise a sample, wherein the sample comprises extracellular nucleic acid within a lipid capsule.

As used herein, “lipid capsule” refers to a lipid structure that entraps or encapsulates the nucleic acid. As would be clear to a person of skill in the art, in the context of the invention, the term “lipid capsule” does not encompass cells themselves (as this is not compatible with the term extracellular nucleic acid). The lipid capsules described herein may therefore be considered as lipid capsules that, unlike a cell, cannot replicate independently. Suitable lipid capsules are well known to a person of skill in the art and include any extracellular lipid structure, including structures comprising lipid bilayers (such as liposomes, enveloped viral particles, lipid bilayer-containing nanoparticles and extracellular vesicles such as exosomes, ectosomes, micro-vesicles, microparticles, oncosomes etc) and structures with lipid monolayers (such as micelles, lipid droplets, lipid monolayer-containing nanoparticles etc). As would be clear to a person skilled in the art, a lipid capsule comprising a lipid monolayer (or lipid bilayer) includes contiguous and non-contiguous lipid monolayers (or contiguous and non-contiguous lipid bilayers) such as those found in some lipid nanoparticles.

In a particular example, lipid capsules include enveloped viral particles.

As used herein, the term “lipid bilayer” refers to a structure comprising two parallel layers of lipid molecules. Generally, each lipid molecule in a lipid bilayer comprises a hydrophilic head and a hydrophobic tail. In a lipid bilayer, the two layers of lipid molecules are arranged such that their hydrophobic tails point inwards towards each other to form a hydrophobic interior and their hydrophilic heads face outwards, towards an aqueous environment. The cell membranes of almost all organisms and many viruses are made of a lipid bilayer, comprising phospholipids, as are the nuclear membrane surrounding the cell nucleus, and membranes of the membrane-bound organelles in the cell. In this context, enveloped viral particles (such as virions) may be considered as having a lipid capsule comprising a lipid bilayer (as the viral particle envelope is typically derived from the host cell during budding).

The term “liposome” as used herein refers to a particle that is prepared from polar lipid molecules derived either from natural sources or chemical synthesis. The lipids form a spherical/oval, closed structure, wherein an external curved lipid bilayer forms around an aqueous core. The liposome may include one or several lipid bilayers enclosing the aqueous core. Liposomes may be used as a vehicle for delivery of a cargo. e.g. a therapeutic agent. For example, liposomes may be used for delivery of pharmaceutical drugs and/or extracellular nucleic acids, including viral vectors. Liposomes may comprise phospholipids, especially phosphatidylcholine, but may also include other lipids, such as phosphatidylethanolamine, so long as they are compatible with lipid bilayer structure. The major types of liposomes are the multilamellar vesicle (MLV, with several lamellar phase lipid bilayers), the small unilamellar liposome vesicle (SUV, with one lipid bilayer), the large unilamellar vesicle (LUV), and the cochleate vesicle. Any suitable liposome may be used in accordance with the present invention. In one example, a reversibly cross-linked hydrogel described herein may therefore comprise a sample, wherein the sample comprises extracellular nucleic acid within a liposome.

In one example, a reversibly cross-linked hydrogel described herein may comprise a sample, wherein the sample comprises extracellular nucleic acid within a lipid-containing nanoparticle. As used herein, the term “lipid-containing nanoparticle” refers to a nanoparticle comprising lipids, optionally with the addition of other components such as proteins. The term “lipid monolayer nanoparticle” as used herein refers to a nanoparticle comprising a lipid monolayer. The term “lipid bilayer nanoparticles” as used herein refers to a nanoparticle comprising a lipid bilayer. As used herein, the term “nanoparticle” refers to particles having at least one dimension on the order of nanometers gr. 1-1,000 nm).

As used herein, “extracellular vesicle” (or “EV”) refers to lipid bilayer-delimited particles that are released from cells and, unlike a cell, cannot replicate. As discussed herein, examples of extracellular vesicles include exosomes, ectosomes, micro-vesicles, microparticles, oncosomes etc. Several extracellular vesicles are known in the art, including exosomes, ectosomes, micro-vesicles, microparticles and oncosomes.

Extracellular vesicles have been well characterised and have a well-defined meaning in the art (reviewed in Andaloussi et al., Nature Reviews Drug Discovery, vol 12, May 2013, page 347-357, Extracellular vesicles; biology and emerging therapeutic opportunities). Extracellular vesicles have been isolated from several bodily fluids. They have been shown to play a key role in the regulation of physiological processes, including stem cell maintenance, immune surveillance and blood coagulation. They have also been shown to play a crucial role in the pathology underlying several diseases.

Extracellular vesicles can be shed from multivesicular bodies (MVBs), which are derived from endosomes, or can bud directly out from the plasma membrane. When they are released into the extracellular space they are referred to as exosomes or ectosomes (or micro-vesicles) depending on whether they have formed from inner or outer cell membranes, and are taken up by other cells through endocytosis or fusion.

Extracellular vesicles are classified according to their cellular origin, biological function, or based on their biogenesis (reviewed in Andaloussi et al., 2013). As determined by their biogenesis, the three main classes of extracellular vesicles are exosomes, micro-vesicles and apoptotic bodies, the first two of which are most predominant in biological samples. EV markers are well known in the art. Examples of exosome markers include tetraspanins (such as TSPAN29 and TSPAN30), ESCRT components, PDCD6IP, TSG101, flotillin, and MFGE8. Examples of micro-vesicle markers include integrins, selectins and CD40 ligand.

Despite recent advances, the terms “exosome” and “micro-vesicle” have been used interchangeably in many published studies. Herein, the term “extracellular vesicle” is used to refer to both vesicle types.

The term “lipid monolayer” as used herein, refers to a membrane comprising a single layer of lipid molecules. Examples of well-known structures with lipid monolayers include micelles, lipid droplets, lipid monolayer nanoparticles etc.

As mentioned elsewhere herein, the extracellular nucleic acid may be entrapped or encapsulated within a lipid capsule. For example, an “entrapped nucleic acid” may be bound to (or adsorbed onto, or tethered to) the outer surface of the lipid capsule. As used herein, the term “entrapped” refers to the extracellular nucleic acid being physically captured/trapped by the lipid capsule, such that it is not released from the lipid capsule. The extracellular nucleic acid may be entrapped by virtue of being completely surrounded by the lipid capsule, or it may be entrapped by virtue of the majority (but not all) of the extracellular nucleic acid being surrounded by the lipid capsule. In this context, the “majority” refers to at least about 50%, at least about 60%, at least about 70%, at least about 80%, at least about 85%, at least about 90%, at least about 95%, at least about 96%, at least about 97%, at least about 98%, or at least about 99% of the extracellular nucleic acid (by sequence) being surrounded by the lipid capsule.

In examples where the extracellular nucleic acid is in a lipid capsule, it is typically encapsulated by the lipid capsule. The term “encapsulated” refers to enclosing the extracellular nucleic acid in the lipid capsule. An extracellular nucleic acid is “encapsulated” by a lipid capsule when it is completely surrounded by the lipid capsule. The extracellular nucleic acid may be located within an aqueous core of the lipid capsule. Alternatively, the extracellular nucleic acid may be located within the lipid structure (e.g. within a lipid bilayer) of the lipid capsule via covalent bonding, electrostatic or hydrophobic interactions. Alternatively, an extracellular nucleic acid (e.g. a hydrophobic drug) may be incorporated into the nanodomain of the lipid capsule.

In some examples, the lipid capsule may include additional cargo (in addition to the extracellular nucleic acid). The additional cargo may also be encapsulated within the lipid capsule. Additional cargo may include a pharmaceutically acceptable carrier, diluent or excipient, or one or more active pharmaceutical ingredients.

In some examples, the extracellular nucleic acid may be present (e.g. adsorbed or absorbed) within or on an absorbent material or fomite (for example when the extracellular nucleic acid is obtained from a subject). In some examples, the absorbent material or fomite may be a swab. In such examples, the absorbent material or fomite may be partially or completely encapsulated in the hydrogel, may be entrapped by the hydrogel, or may be partially or completely immersed in the hydrogel. Typically, the absorbent material or fomite is contacted with the hydrogel forming polymer before or during polymerisation such that at least a portion of the absorbent material or fomite (in or on which the nucleic acid is present) is within the hydrogel once the hydrogel has been formed.

In one example, the sample that is comprised within the reversibly cross-linked hydrogel comprises an aqueous buffer in addition to the extracellular nucleic acid. The terms “aqueous buffer” and “buffer solution” are used interchangeably herein. The aqueous buffer is a pH buffer (or a hydrogen ion buffer). A buffer solution is one which resists changes in pH when small quantities of an acid or an alkali are added to it. Buffer solutions are used as a means of keeping pH at a nearly constant value in a wide variety of chemical applications.

Several appropriate aqueous buffers are known to a person of skill in the art. As a non-limiting example, the aqueous buffer may be a salt-based buffer, for example, a 0.1% to 2.0% (w/v) salt-based buffer. Any suitable salt-based buffer may be used and such buffers are readily identifiable by a person of skill in the art.

In some examples, the aqueous buffer (e.g. the salt-based buffer) may include serum such as fetal calf serum (FCS) or fetal bovine serum (FBS) and/or may include, additional components, such as antibiotics. The addition of FBS or FCS may be useful, for example, when the extracellular nucleic acid is within a lipid capsule, as the serum may help to protect the lipid membrane. Suitable serum and/or antibiotic concentrations may be readily identifiable to a person of skill in the art.

Non-limiting examples of suitable salt-based buffers include Hank's balanced salt solution (or Hank's buffered saline solution), phosphate-buffered saline, sodium chloride or others. As would be clear to a person of skill in the art, any of these buffers can be used as an additional reagent within the sample (to assist with maintaining the pH of the sample in which the nucleic acid is present). A further example of a suitable salt-based buffer is the widely used viral transport medium, Virocult®.

Accordingly, in one example, a reversibly cross-linked hydrogel described herein may comprise a sample, wherein the sample comprises extracellular nucleic acid (e.g. within a lipid capsule such as an enveloped viral particle) and a salt-based solution. For example, the reversibly cross-linked hydrogel may comprise a sample, wherein the sample comprises extracellular nucleic acid (e.g. within a lipid capsule such as an enveloped viral particle) and a viral transport medium (e.g. Virocult®). The salt-based solution (e.g. viral transport medium such as Virocult®, or similar) may be an about 0.1% to about 2.0% (w/v) salt-based solution, for example, the salt-based solution may be an about 0.4% to about 0.9% (w/v) salt-based solution.

As used herein a “reversibly cross-linked hydrogel” refers to a hydrogel that is formed by reversible cross-linking (i.e. the cross-linking can be reversed such that the hydrogel reverts back to a solution). Reversal of the cross-linking enables the extracellular nucleic acid to be released from the hydrogel (e.g. at the point of use/after transportation or storage is complete). Examples of reversibly cross-linked hydrogels are well known in the art. Accordingly, suitable hydrogels may readily be identified by a person of skill in the art.

The hydrogel referred to herein comprises a hydrogel-forming polymer (i.e. at least one hydrogel forming polymer) having a cross-linked or network structure or matrix; with an interstitial liquid. The hydrogel is capable maintaining the structural integrity and/or functionality of the extracellular nucleic acid contained within. Preferably, the hydrogel is semi-permeable.

The term “hydrogel-forming polymer” refers to a polymer which is capable of forming a cross-linked or network structure or matrix under appropriate conditions, wherein an interstitial liquid and an extracellular nucleic acid may be retained within such a structure or matrix. The hydrogel may comprise internal pores.

Initiation of the formation of the cross-linked or network structure or matrix may be by any suitable means, depending on the nature of the polymer.

The polymer will in general be a hydrophilic polymer. It will be capable of swelling in an aqueous liquid. In one example, the hydrogel-forming polymer is collagen. In this example, the collagen hydrogel comprises a matrix of collagen fibrils which form a continuous scaffold around an interstitial liquid and the extracellular nucleic acid. Dissolved collagen may be induced to polymerise/aggregate by the addition of dilute alkali to form a gelled network of cross-linked collagen fibrils. The gelled network of fibrils supports the original volume of the dissolved collagen fibres, retaining the interstitial liquid. General methods for the production of such collagen gels are well known in the art (e.g. WO2006/003442, WO2007/060459 and WO2009/004351).

The collagen which is used in the collagen gel may be any fibril-forming collagen.

Examples of fibril-forming collagens are Types I, II, Ill, V, VI, IX and XI. The gel may comprise all one type of collagen or a mixture of different types of collagen. Preferably, the gel comprises or consists of Type I collagen. In some examples of the invention, the gel is formed exclusively or substantially from collagen fibrils, i.e. collagen fibrils are the only or substantially the only polymers in the gel. In other examples of the invention, the collagen gel may additionally comprise other naturally-occurring polymers, e.g. silk, fibronectin, elastin, chitin and/or cellulose. Generally, the amounts of the non-collagen naturally-occurring polymers will be less than 5%, preferably less than 4%, 3%, 2% or 1% of the gel (wt/wt). Similar amounts of non-natural polymers may also be present in the gel, e.g. peptide amphiphiles, polylactone, polylactide, polyglycone, polycaprolactone and/or phosphate glass.

In some examples, the hydrogel-forming polymer is alginic acid or an alginate salt of a metal ion. Preferably, the metal is a Group 1 metal (e.g. lithium, sodium, or potassium alginate) or a Group 2 metal (e.g. calcium, magnesium, barium or strontium alginate). Preferably, the polymer is calcium alginate or sodium alginate or strontium alginate, most preferably calcium alginate.

One factor which determines alginate gel permeability is the mannuronic (M) and guluronic (G) acid contents of the gel. Gels with a high M:G ratio have a small intrinsic pore size. The M:G ratio may be manipulated to increase the permeability of gels as necessary to improve the preservation of the extracellular nucleic acid within the hydrogel. In some examples, the G content of the alginate gel is 0-30%. In some examples, the M content is preferably 30-70%. In some preferred examples, the gel is an alginate gel with a M content of 50-70% or and the gel additionally comprises or a pore enhancer (also referred to herein as a porogen). In some examples, the pore size increasing agent is hydroxyethyl cellulose (HEC). In this example, HEC may be used in the preparation of the hydrogel; it is then completely, substantially completely or partially removed from the hydrogel prior to use. Preferred concentrations of HEC in the hydrogel (during preparation) include 0.5-3.0% HEC, more preferably 1.0-2.5%, and even more preferably 1.2-2.4% HEC. In some preferred embodiments, the concentration of HEC in the hydrogel (during preparation) is 1.2% or 2.4%. (Concentrations are given as weight %). The HEC may be suspended in the gels as micelles. Removal of the HEC may be attained by washing the hydrogel in a suitable aqueous solvent or buffer, e.g. tissue culture medium.

In some examples, the hydrogel-forming polymer is an alginate. In some examples, the extracellular nucleic acid can be coated first with a different hydrogel-forming polymer as described herein followed by a further coating of an alginate. In other examples, the hydrogel-forming polymer is a mixture of alginate and another hydrogel-forming polymer. In some examples, the alginate is modified (e.g. with peptides).

In yet other examples, the hydrogel-forming polymer is a cross-linked acrylic acid-based (e.g. polyacrylamide) polymer.

In yet further examples, the hydrogel-forming polymer is a cross-linkable cellulose derivative, a hydroxyl ether polymer (e.g. a poloxamer), pectin or a natural gum.

In some examples, the hydrogel is not thermo-reversible at physiological temperatures, i.e. the sol-gel transition of the hydrogel cannot be obtained at a temperature of 0-40° C.

The structure of the hydrogel may be changed by varying the concentration of the hydrogel-forming polymer in the hydrogel. The structure affects the robustness of the gel and its handling properties. Preferred concentrations of the hydrogel-forming polymer in the hydrogel are 0.1-5% (weight of polymer to volume of interstitial liquid), and include for example 0.1-0.4%, 0.2-0.4%, 0.4-0.5%, 0.5-0.7%, 0.7-1.1%, 1.1-1.3%, 1.3-2.2%, 2.2-2.6%, 2.6-3.0%, 3.0-3.5%, 3.5-4.0%, 4.0-4.5% and 4.5-5.0% (or any combination thereof e.g. 0.1-0.5%, 0.2 to 0.7% etc).

In one example, the viscosity of the non-gelled hydrogel solution is up to 500 mPa·s, Optionally, the viscosity of the non-gelled hydrogel solution is between 5 and 200 mPa·s (preferably between 5 and 100 mPa.$).

In other examples, the concentration of the hydrogel-forming polymer in the hydrogel is above 0.3%, 0.4%, 0.5% or 0.6%. In other examples, the concentration of the hydrogel-30 forming polymer in the hydrogel is below 5%, 4.5%, 4.0%, 3.5%, 3.0%, 2.6%, 2.4%, 1.5%, 1.4%, 1.3% or 1.2%. In some preferred examples, the concentration of the hydrogel-forming polymer in the hydrogel is about 0.3%, about 0.6% or about 1.2%. In some particularly preferred examples, the concentration of the hydrogel-forming polymer in the hydrogel is about 1%. In some particularly preferred examples, the hydrogel is formed from about 1% sodium 35 alginate or from about 1% calcium alginate.

In some examples, the gelling of the hydrogel is facilitated using a compound comprising a multivalent metal cation, e.g. using calcium chloride. In particular, calcium chloride (e.g. 50-200 mM calcium chloride, preferably 75-120 mM calcium chloride) may be used to gel alginate hydrogels.

In other examples, an alternative metal chloride is used, e.g. magnesium or barium or strontium chloride. Alternatively, other multivalent cations may be used, e.g. La³⁺ or Fe³⁺.

The invention further provides a process for preparing a hydrogel, comprising the step of gelling the hydrogel-forming polymer in the presence of a Group 2 metal salt selected from the group consisting of magnesium and calcium salts.

In some examples, the hydrogel comprises cross-linked alginate. For example, the hydrogel may comprise cross-linked calcium-alginate, strontium-alginate, barium-alginate, magnesium-alginate and/or sodium-alginate.

In one example, the hydrogel may comprise cross-linked calcium-alginate, optionally wherein the hydrogel comprises cross-linked calcium-alginate and sodium-alginate. Accordingly, in one example, a reversibly cross-linked calcium-alginate hydrogel (optionally with sodium-alginate) is provided, wherein the hydrogel comprises a sample, wherein the sample comprises extracellular nucleic acid.

In any of the examples described herein, the cross-linked alginate may be from about 0.1% (w/v) to about 5.0% (w/v) calcium alginate. For example, the cross-linked alginate may be from about 0.5% (w/v) to about 3.0% (w/v), 1.0% (w/v) to about 2.5% (w/v), about 1.5% (w/v) to about 2.0% (w/v) calcium alginate, or any range therebetween.

The interstitial liquid may be any liquid in which polymer may be dissolved and in which the polymer may gel. Generally, it will be an aqueous liquid, for example an aqueous buffer. Suitable aqueous buffers are described elsewhere herein. The liquid may contain an antibiotic. Preferably, the hydrogel is sterile, i.e. aseptic.

The hydrogels may be produced in any suitable size.

In some examples, the hydrogel is in the form of a thin layer, disc or sheet. Preferably, the gel is in the form of a disc or thin layer. The disc may for example, have a diameter of 5-50 mm or 10-50 mm, preferably 10-30 mm, more preferably 15-25 mm, and most preferably about 19 mm. The thickness of the thin layer, disc or sheet is generally 0.1-5 mm, preferably 0.5-2.0 mm, more preferably about 1.0 or 1.5 mm, or about 1, 2, 3, 4 or 5 mm. In some examples, the final volume of hydrogel in the disc is preferably 200 μl to 1 ml, preferably 200-600 μl, preferably 300-500 μl and more preferably 400-450 μl.

The hydrogel described herein any be formed using any suitable means. As described in the example section below, one non-limiting means for forming the hydrogels described herein in is the use of reagents available in the field, for example SwabReady™. In this example, gel beads comprising calcium-alginate are used and additional alginate is added with the sample to induce further crosslinking. These hydrogels comprise two hydrogel forming polymers (sodium alginate and calcium alginate). Such hydrogels are clearly encompassed by the claims, as they comprise a reversibly cross-linked hydrogel.

As described in detail below, the reversibly cross-linked hydrogel (comprising the extracellular nucleic acid) may be packaged in a sealed receptacle.

As used herein, a “sealed receptacle” refers to a container that can maintain a seal against the continuous flow of gases or liquids. For example, the sealed receptacle may be a water-tight and/or air-tight container e.g. a plastic container. Non-limiting examples of appropriate sealed receptacles include a sealed vial or cryovial or tissue culture flask, optionally together with an appropriate buffer (e.g. a salt based buffer). In other examples, the hydrogel may be contained within a sealed bag.

In one example, the sealed receptacle is selected from a tube, a flask, a dish, a vessel or a plate (e.g. a plate comprising a plurality of wells). For example, the plate may be selected from a 4-, 6-, 8-, 12-, 24-, 48-, 96-, 384-, 1536-well plate. Appropriate receptacles are well known in the art.

The receptacle may be sealed using a lid (e.g. a screw fit lid) or another means (e.g. adhesive film, or tape etc).

The inventors have surprisingly shown that incorporation of extracellular nucleic acid into a reversibly cross-linked hydrogel protects the extracellular nucleic acid from the mechanical and environmental stresses of storage and preserves the structural integrity and/or functionality of the extracellular nucleic acid. The reversibly cross-linked hydrogels described herein may be used to preserve extracellular nucleic acid (i.e. by maintaining its structural integrity and/or functionality during periods of storage and/or transportation).

As used herein, the phrase “maintaining the structural integrity of the extracellular nucleic acid” means that all or a substantial portion of the nucleic acid is unchanged during the specified period of storage and/or transportation in the hydrogel (compared to the structural integrity of the nucleic acid before being contacted with the hydrogel). Similarly, the phrase “maintaining the functionality of the extracellular nucleic acid” means that a substantial portion of the original function of the nucleic acid is maintained during the specified period of storage and/or transportation in the hydrogel (where the original function is measured as the function before being contacted with the hydrogel). A substantial proportion may be at least 50%, 60%, 70%, 80%, 90% or 95%. Suitable assays for determining the structural integrity and functionality of nucleic acids are well known in the art. For example, nucleic acid quantification assays such as qPCR may be used to quantify the amount of nucleic acid that has been preserved over the specified period of time. The preservation of sequence integrity can also be assessed using next generation sequencing or, pyrosequencing. As another example, assays that determine viral cytopathic effect (CPE) may be used (e.g. when the nucleic acid is within a viral vector, viral particle or virion). Further details of such assays are provided in the “examples” section below.

Methods of Preparing a Sample Comprising Extracellular Nuclear Acid for Storage and/or Transportation

The invention also provides a method of preparing a sample comprising extracellular nucleic acid for storage or transportation from a first location to a second location. The method comprises the steps of:
i) contacting the sample with a hydrogel-forming polymer; and
ii) polymerising the polymer to form a reversibly cross-linked nucleic acid-containing hydrogel.

In one example, the method may comprise mixing the extracellular nucleic acid (e.g. an extracellular nucleic acid within a lipid capsule, such as an enveloped viral particle) with an aqueous buffer prior to step (i). Several appropriate buffers are described elsewhere herein and apply equally here. For example, the method may comprise mixing the extracellular nucleic acid (e.g. an extracellular nucleic acid within a lipid capsule, such as an enveloped viral particle) with a salt-based buffer prior to step (i).

The method may further comprise sealing the nucleic acid-containing hydrogel into a receptacle for storage or transportation from the first location to the second location. The receptacle within which the nucleic acid-containing hydrogel was formed may be a receptacle that is suitable for storage or transportation from the first location to the second location—in this example, the method may merely comprise sealing the hydrogel into the receptacle in which it was formed. In another example, the method may comprise placing the formed hydrogel into a suitable receptacle, before sealing the receptacle. In this context, the method may comprise packaging and sealing the nucleic acid-containing hydrogel into the receptacle for storage or transportation from the first location to the second location. Examples of suitable receptacles are discussed elsewhere and include a vial, tube, flask, dish, vessel or plate. Several means for sealing the nucleic acid-containing hydrogel into the receptacle are known in the art (e.g. the using a lid, adhesive film, or tape etc).

Accordingly, the extracellular nucleic acid may be placed within the receptacle prior to step i) of the method e.g. the hydrogel-forming polymer may be contacted with the extracellular nucleic acid whilst the extracellular nucleic acid is located within the receptacle that is suitable for storage or transportation.

Alternatively, the extracellular nucleic acid may be placed within the receptacle after step (i) of the method e.g. the hydrogel-forming polymer may be contacted with the extracellular nucleic acid (and optionally polymerised as per step ii)) before the extracellular nucleic acid is placed within the receptacle that is suitable for storage or transportation.

A method of preparing a sample comprising extracellular nucleic acid (e.g. an extracellular nucleic acid within a lipid capsule such as a viral particle) for storage or transportation from a first location to a second location is therefore provided, the method comprising the steps of:
i) contacting the sample with a hydrogel-forming polymer; and
ii) polymerising the polymer to form a reversibly cross-linked nucleic acid-containing hydrogel and sealing the nucleic acid-containing hydrogel into a receptacle suitable for storage or transportation from a first location to a second location.

The method may further comprise iii) dispatching the sealed receptacle for transportation from the first location to the second location. As used herein, “dispatching” refers to releasing the receptacle for transport (e.g. releasing the receptacle to the courier for transport/delivery to the intended destination). Dispatch therefore does not include transport of the sealed receptacle to the second location per se.

The extracellular nucleic acid may be contacted with a hydrogel-forming polymer using any appropriate means. For example, extracellular nucleic acid may be mixed with a solution that contains the hydrogel forming polymer (prior to polymerization/aggregation or prior to cross-linking of a hydrogel-forming polymer). As would be clear to a person of skill in the art, contacting with “a hydrogel-forming polymer” encompasses contacting the extracellular nucleic acid one hydrogel-forming polymer, or more than one (e.g. two) hydrogel-forming polymers. For example, the sample may be contacted with strontium alginate and calcium alginate during formation of the hydrogel.

The extracellular nucleic acid may be contacted with the hydrogel-forming polymer whilst within a sealable receptacle (such that e.g. once the hydrogel is formed, the receptacle can be sealed ready for storage and/or transportation), or it may be contacted with the hydrogel-forming polymer before the extracellular nucleic acid is placed in a sealable receptacle. Suitable receptacles are described elsewhere herein.

The method then comprises polymerising the extracellular nucleic acid-polymer to form a reversibly cross-linked nucleic acid-containing hydrogel wherein the extracellular nucleic acid is within the hydrogel. Methods for polymerising the extracellular nucleic acid-polymer to form a reversibly cross-linked nucleic acid-containing hydrogel are well known in the art, and differ depending on the polymer used. For example, polymerisation of an alginate solution (to form an alginate hydrogel) may be induced by a chemical agent such as calcium chloride.

As used herein, the terms “polymerising” and “gelling” the hydrogel are used interchangeably to refer to the change in state of the hydrogel-forming polymer from a liquid to a hydrogel.

The hydrogel is gelled under appropriate cell-compatible conditions, i.e. conditions which are not detrimental or not significantly detrimental to the structural integrity and/or functionality of the extracellular nucleic acid.

In some examples, the hydrogels are prepared under cGMP (current Good Manufacturing Practice) conditions.

Methods of transporting/storing/fulfilling an order for an extracellular nucleic acid A method of transporting a sample comprising an extracellular nucleic acid from a first location to a second location is also provided herein. The method comprises the steps of:
(a) obtaining a reversibly cross-linked nucleic acid-containing hydrogel generated according to the methods described elsewhere herein; or obtaining a reversibly cross-linked nucleic acid-containing hydrogel described elsewhere herein;
(b) transporting the hydrogel from the first location to the second location; and optionally
(c) releasing the nucleic acid from the hydrogel at the second location.

Aspects of the invention described elsewhere (e.g. suitable receptacles, hydrogels, nucleic acids, buffers) apply equally here.

Furthermore, a method of storing a sample comprising an extracellular nucleic acid is also provided, the method comprising the steps of:
(a) obtaining a reversibly cross-linked nucleic acid-containing hydrogel generated according to the methods described elsewhere herein; or obtaining a reversibly cross-linked nucleic acid-containing hydrogel described elsewhere herein; and
(b) storing the hydrogel.

Aspects of the invention described elsewhere (e.g. suitable receptacles, hydrogels, nucleic acids, buffers) apply equally here.

The method may be used to store and/or transport the nucleic acid-containing hydrogel for any suitable period of time. For example, the nucleic acid-containing hydrogel may be stored and/or transported for at least 6 hours, at least 12 hours, at least 24 hours. Typically, the nucleic acid-containing hydrogel may be stored and/or transported for at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days etc. For example, the nucleic acid-containing hydrogel may be stored and/or transported for at least 3 days, or at least 5 days. The nucleic acid-containing hydrogel may be stored and/or transported for at least 7 days etc.

The extracellular nucleic acid-containing hydrogels may be stored and/or transported for up to or 20 weeks. Preferably, the extracellular nucleic acid is stored in the hydrogel for up to 1, 2, 3, 4, 5, 6, 7, 8, 9 or 10 weeks before being released from the hydrogels. More preferably, the extracellular nucleic acid is stored in the hydrogel for up to 1, 2, 3, 4, 5, 6, 7, 8, 9 or 10 days before being released from the hydrogels.

In one example, wherein the extracellular nucleic acid is within a lipid capsule, such as an enveloped viral particle, the method may be used to store and/or transport the nucleic acid-containing hydrogel for any suitable period of time. For example, the nucleic acid-containing hydrogel may be stored and/or transported for at least 6 hours, at least 12 hours, at least 24 hours. Typically, the nucleic acid-containing hydrogel may be stored and/or transported for at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days etc. For example, the nucleic acid-containing hydrogel may be stored and/or transported for at least 3 days, or at least 5 days. The nucleic acid-containing hydrogel may be stored and/or transported for at least 7 days etc.

The extracellular nucleic acids described herein may be transported within the hydrogel (and sealed receptacle) by any suitable means, e.g. by post or courier, which might include transportation by automotive means, e.g. by car, van, lorry, motorcycle, aeroplane, etc. Preferably, the transportation is by post or courier.

The second location is preferably a location which is remote from the first location, e.g. at least 1 mile, preferably more than 5 miles, from the first location.

Transportation from a first location to a second location may take at least 1 hour, at least 2 hours, at least 5 hours, at least 12 hours, at least 24 hours etc.

Appropriate conditions for storing and/or transporting the nucleic acid-containing hydrogels described herein may be readily identified by a person of skill in the art, and include maintaining the nucleic acid-containing hydrogels at a desired temperature (e.g. at between 18 to 22° C., as used in the examples). For example, appropriate conditions for storing and/or transporting a nucleic acid-containing hydrogel wherein the nucleic acid is within a lipid capsule such as an enveloped viral particle include maintaining the nucleic acid-containing hydrogels at a desired temperature (e.g. at between 18 to 22° C., as used in the examples).

The extracellular nucleic acids may be stored and/or transported within the hydrogel (and the sealed receptacle) at a temperature ranging from −80° C. to 45° C., preferably at 4 to 45° C. in one example, the extracellular nucleic acid is stored and/or transported at ambient temperature.

In some examples, the extracellular nucleic acids within the hydrogels (and sealed receptacle) are stored and/or transported under chilled conditions, e.g. 4-6° C., preferably about 4° C. In a particular example, they are refrigerated when stored and/or transported (which is defined as from 2-8° C. (EU Pharmacopoeia)). In another example, they are stored and/or transported cool (defined as from 8-15° C.)).

In other examples, they are stored and/or transported under ambient conditions, e.g. 10-25° C., preferably 15-22° C., e.g. 18 to 22° C. In some examples, the ambient temperature may be up to 30° C. (i.e. 10 to 30° C.), or even up to 40° C. In yet other examples, they are stored and/or transported at about 37° C.

In some examples, they are stored and/or transported at Controlled Room Temperature (CRT) (which is defined as from 15 to 25° C.). They may be stored or transported cool or at CRT (i.e. from 8 to 25° C.).

In yet other examples, they are stored and/or transported at hypothermic temperatures (i.e. below about 35° C., typically in the range of 0 to 32° C.). In one example, they are stored and/or transported between CRT and 32° C. (i.e. 15 to 32° C.). In another example, they are stored and/or transported cool, at CRT or up to 32° C. (i.e. from 8 to 32° C.).

In some examples, the hydrogel comprising the extracellular nucleic acid is frozen prior to storage and/or transportation. This may extend the time during which the extracellular nucleic acid maintains its structural integrity and/or functionality post-thawing and/or may increase the usable transit-time. Hence the hydrogel may be used in this way as a post-cryoprotectant. For example, the temperature of the hydrogel comprising the extracellular nucleic acid may be reduced to below 0° C., below −15° C. or below −80° C. The hydrogel comprising the extracellular nucleic acid may or may not be allowed to defrost or thaw, i.e. to increase its temperature to above 0° C. during storage and/or transportation, preferably at a slow, controlled or uncontrolled rate of temperature increase. In other examples the hydrogels of the invention are not chilled or frozen.

A method for fulfilling an order or request for an extracellular nucleic acid is also provided, the method comprising the steps of:
a) receiving an order or request for an extracellular nucleic acid;
b) obtaining a reversibly cross-linked nucleic acid-containing hydrogel generated according to the methods described elsewhere herein; or obtaining a reversibly cross-linked nucleic acid-containing hydrogel as described elsewhere herein; and
c) dispatching the sample for transportation; or transporting the sample to the location specified in the order or request.

The order or request may be received by any suitable means, e.g. via the internet, email, text-message, telephone or post.

Aspects of the invention described elsewhere (e.g. suitable receptacles, hydrogels, nucleic acids, buffers) apply equally here.

The hydrogel referred to herein is one from which the extracellular nucleic acid can be released. In other words, after the preservation or storage and/or transport of the extracellular nucleic acid contained therein, the hydrogel is capable of being dissociated thus allowing the release or removal of all or substantially all of extracellular nucleic acid which was previously retained therein.

The hydrogel is dissociated under appropriate nucleic acid-compatible conditions, i.e. conditions which are not detrimental or not significantly detrimental to the nucleic acid and/or the integrity of the lipid capsule within which the nucleic acid may be located.

Preferably, the hydrogel is dissociated by being chemically disintegrated or dissolved. For example, alginate gels may be disintegrated in an appropriate alginate dissolving buffer (e.g. 0.055 M sodium citrate, 0.15 M NaCl, pH 6.8). Another suitable alginate dissolving buffers are well known to a person skilled in the art.

Preferably, at least 50%, 60% or 70% of the extracellular nucleic acid retains its structural integrity and/or functionality after storage and/or transport, more preferably at least 80%, 85%, 90% or 95% of the extracellular nucleic acid retains its structural integrity and/or functionality after storage and/or transport. Assays for assessing structural integrity and/or functionality of the extracellular nucleic acid are discussed elsewhere herein.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

Materials

Cell types: MRC-5 (ATCC® CCL-171™)

Virus: Human coronavirus 229E (CoV 229E) (ATCC® VR-740™)

TABLE 1
Test agents used throughout the study.

| Test agent | Test agent format | Test agent description |
|---|---|---|
| Atelerix SwabReady ™ | In situ curing gel | Hydrogel |
| Virocult ® viral | Liquid | Viral transport medium |
| transport medium | | |

Reagents:

GelBase Beads: 0.55% Calcium Alginate Beads

Atelerix SwabReady™ modified viral transport medium: 1% Sodium Alginate solution in Virocult medium

Methods

SwabReady™ Manufacturing Summary:

1) Powdered low sodium alginate is combined with Hank's buffered saline solution to a concentration of 0.55%.

2) 0.4 mL of this 0.55% sodium alginate solution is added dropwise into 1 mL of 0.1 molar calcium chloride solution. The divalent cations, Ca2+, in the calcium chloride crosslink the carboxylate groups of the guluronate groups in the polymer backbone, forming gel beads. This is incubated overnight allowing the process to go to completion. After which the solution of calcium chloride is removed and the beads are washed with saline solution. This forms our Calcium Alginate Beads.

3) Powdered sodium alginate is combined with Hank's buffered saline solution to a concentration of 5%.

4) 0.1 mL of the 5% sodium alginate solution is added to 0.4 mL of Virocult medium to create a 1% solution of Atelerix SwabReady™ modified viral transport medium.

5) 0.2 Molar tri-sodium citrate is dissolved in phosphate buffered saline solution to create our dissolution buffer.

SwabReady™ Usage Summary:

1) 0.5 mL Atelerix SwabReady™ modified viral transport medium is added to 0.4 mL 0.55% Calcium Alginate Beads (GelBase beads). The 0.55% beads donate some of their divalent cations, Ca2+, to the carboxylate groups of the guluronate groups in the polymer backbone of the 1% solution of Atelerix SwabReady™ modified viral transport medium.

2) Whilst still liquid a used Swab can be inserted into the gel solution, gelation/crosslinking usually takes 2 hours.

3) The swab kit can now be transported to a laboratory for testing

4) To release the swab from the gel, 0.3 mL of the dissolution buffer is added to the tube. The tri-sodium citrate removes the calcium cations from the polymer backbone, reverting the crosslinking process.

Cell Maintenance and Assay Set-Up

MRC-5 cells were used as the host cell line for human coronavirus 229E (CoV 229E) propagation. MRC-5 cells were maintained in Eagle's Minimum Essential Medium (EMEM; ATCC®, UK) supplemented with 20% Foetal Bovine Serum (FBS) (Gibco™, USA) and 1% penicillin-streptomycin (ThermoFisher Scientific, UK) (complete culture medium) at 37±2° C. and 5% CO₂. In preparation for the viral titration, MRC-5 cells were seeded into 96 well plates at 2×10⁵ cellsmL⁻¹ and incubated at 37±2° C. and 5% CO₂ for 24 hours or until 80-90% confluency was reached.

HeLa cells were used as the host cell lines for Adenovirus type 5 propagation. HeLa cells were maintained in Eagle's Minimum Essential Medium (EMEM) supplemented with 10% Foetal Bovine Serum (FBS) and 1% penicillin-streptomycin (complete culture medium) at 37±2° C. and 5% CO₂. In preparation for the viral titration, HeLa cells and were seeded into 96 well plates and incubated at 37±2° C. and 5% CO₂ for 24 hours or until 80-90% confluency was reached.

SCP cells were used as the host cell line for Visna virus propagation. The cells were maintained in Eagle's Minimum Essential Medium (EMEM), supplemented with 10% Foetal Bovine Serum (FBS) and 1% penicillin-streptomycin (complete culture medium) at 37±2° C. and 5% CO₂. In preparation for the viral titration, SCP cells and were seeded into 96 well plates and incubated at 37±2° C. and 5% CO₂ for 24 hours or until 80-90% confluency was reached.

Viability Assay Set-Up

The evaluation of viability of cells and human coronavirus was performed following manufacturer's instructions for the use of Atelerix SwabReady™ and Virocult® viral transport medium. Testing was performed in triplicate and conducted at a controlled room temperature of 20±2° C. An aliquot of 200 μL of a 5.5×10 5 TCID₅₀/mL human coronavirus 229E was added to 500 μL of Virocult®. An aliquot of 500 μL of Atelerix SwabReady™ modified viral transport medium was added to GelBase Beads and the tubes were inverted vigorously to distribute the beads throughout the modified viral medium. Once fully mixed, the contents were allowed to settle to the bottom of the tube and 200 μL of a 5.5×10 5 TCID₅₀/mL human coronavirus 229E was added to each tube human coronavirus 229E was added within 5 minutes of the addition of the modified viral medium. Samples were incubated at 20±2° C. within a laminar flow cabinet for up to 14 days.

Viral Viability Quantification by TCID50

Human coronavirus 229E (CoV 229E):

Following preparation of the test samples as per the “viability assay set up” above, 10-fold serial dilutions were performed in EMEM containing 2% FBS and 1% penicillin-streptomycin (assay medium). Medium was aspirated from the wells of a pre-seeded MRC-5 cell plate and cells were washed with Dulbecco's Phosphate buffered saline (DPBS; Gibco™, UK) containing calcium and magnesium. An aliquot of 100 μL of each dilution of the test samples were added to the corresponding test wells. Test plates were incubated at 35±2° C. and 5% CO₂ for 7 days. Six replicates wells were performed for each test replicate. After incubation, viral cytopathic effect (CPE) was determined using an Motic AE 2000 inverted microscope. The viral titre was calculated using the Spearman-Kärber method.

Adenovirus:

Samples were analysed following 0, 3, 7 and 14 days incubation. An aliquot of 400 μL of SwabReady™ dissolution buffer was added to Atelerix Swabready™ samples and incubated for 15 minutes to allow the gel to dissolve. A 100 μL aliquot from the Atelerix Swabready™ samples and the PBS controls was taken and diluted 10-fold in EMEM supplemented with 2% FBS and 1% penicillin-streptomycin (complete assay medium). Medium was aspirated from the wells of pre-seeded HeLa cell plates and cells were washed with Dulbecco's Phosphate buffered saline (DPBS) containing calcium and magnesium. A 100 μL aliquot of each dilution was added to the corresponding cell plates. Test plates were incubated at 37±2° C. and 5% CO₂ for 5 days. Eight replicate wells were performed for each test replicate. After incubation, viral cytopathic effect (CPE) was determined using an Motic AE 2000 inverted microscope. The viral titre was calculated using the Spearman-Kärber method.

Visna Virus:

Samples were analysed following 3, 7 and 14 day incubation. An aliquot of 400 μL of SwabReady™ dissolution buffer was added to Atelerix Swabready™ samples and incubated for 15 minutes to allow the gel to dissolve. A 100 μL aliquot from the Atelerix Swabready™ samples and the PBS controls was taken and diluted 10-fold in EMEM supplemented with 2% FBS, 1% penicillin-streptomycin and 1% L-Glutamine (complete assay medium). Medium was aspirated from the wells of pre-seeded SCP cell plates and cells were washed with Dulbecco's Phosphate buffered saline (DPBS) containing calcium and magnesium. A 100 μL aliquot of each dilution was added to the corresponding cell plates. Eight replicate wells were performed for each test replicate and plates were incubated at 37±2° C. and 5% CO₂ for 7 days. After incubation, viral cytopathic effect (CPE) was determined using a Motic AE 2000 inverted microscope. The viral titre was calculated using the Spearman-Kärber method.

Results

Viral Preservation Efficacy of a Hydrogel-Based Sample Storage Technology

Human Coronavirus 229E

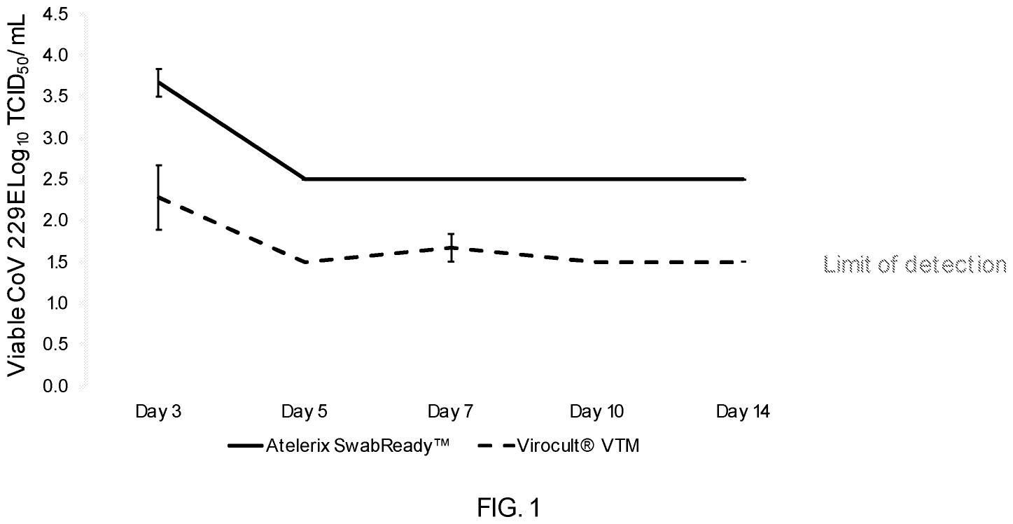

After 3 days incubation, an average of 3.67 Log 10 TCID₅₀/mL viable CoV 229E was recovered from Atelerix SwabReady™ hydrogel storage. An average of 2.28 Log 10 TCID₅₀/mL viable CoV 229E was recovered from Virocult® viral transport medium. Following 5 days, 7 days, 10 days and 14 days incubation, an average of 2.5 Log 10 TCID₅₀/mL viable CoV 229E was recovered from Atelerix SwabReady™ hydrogel storage. After 7 days, an average of 1.67 Log 10 TCID₅₀/mL viable CoV 229E was recovered from Virocult® viral transport medium. After days, 10 days and 14 days, less than 1.50 Log 10 TCID₅₀/mL viable CoV 229E was recovered from Virocult® viral transport medium as no viral CPE was observed. (Table 2, FIG. 1).

TABLE 2
Log TCID50/mL for human coronavirus 229E (CoV 229E)
following incubation in Atelerix SwabReady ™ and
Virocult ® viral transport medium for up to 14 days.

| | Average viable CoV 229E | | | | |
|---|---|---|---|---|---|
| | (Log TCID50/mL) | | | | |
| Treatment | Day 3 | Day 5 | Day 7 | Day 10 | Day 14 |
| Atelerix SwabReady ™ | 3.67 | 2.50 | 2.50 | 2.50 | 2.50 |
| Virocult ® | 2.28 | <1.50 | 1.67 | <1.50 | <1.50 |

Adenovirus

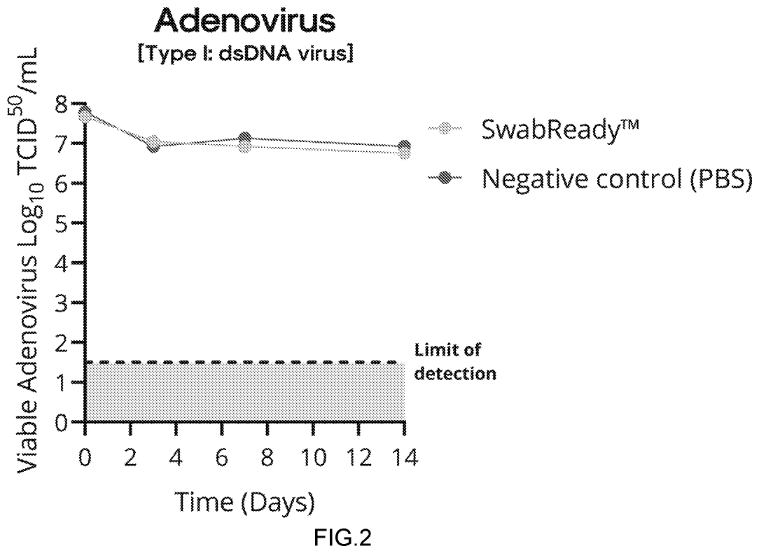

After 0 days incubation, an average of 7.67 Log TCID₅₀ mL-1 viable Adenovirus was recovered from Atelerix SwabReady™ and an average of 7.79 Log TCID₅₀ mL-1 viable Adenovirus was recovered from the PBS control. Following 3 days, 7 days and 14 days incubation, an average of 7.04, 6.92 and 6.75 Log TCID₅₀ mL-1 viable Adenovirus was recovered respectively from Atelerix SwabReady™. Following 3 days, 7 days and 14 days incubation, an average of 6.92, 7.13 and 6.92 Log TCID₅₀ mL-1 viable Adenovirus was recovered respectively from the PBS control (Table 3, FIG. 2).

TABLE 3
Average LogTCID₅₀ mL−1 recovery for Adenovirus
following incubation in Atelerix SwabReady ™ and
Phosphate buffered saline for up to 14 days.

| | Average viable Adenovirus type 5 | | | |
|---|---|---|---|---|
| | (LogTCID₅₀ mL⁻¹) | | | |
| Test agent | Day 0 | Day 3 | Day 7 | Day 14 |
| Atelerix SwabReady ™ | 7.67 | 7.04 | 6.92 | 6.75 |
| Phosphate buffered | 7.79 | 6.92 | 7.13 | 6.92 |
| saline control | | | | |

Visna Virus

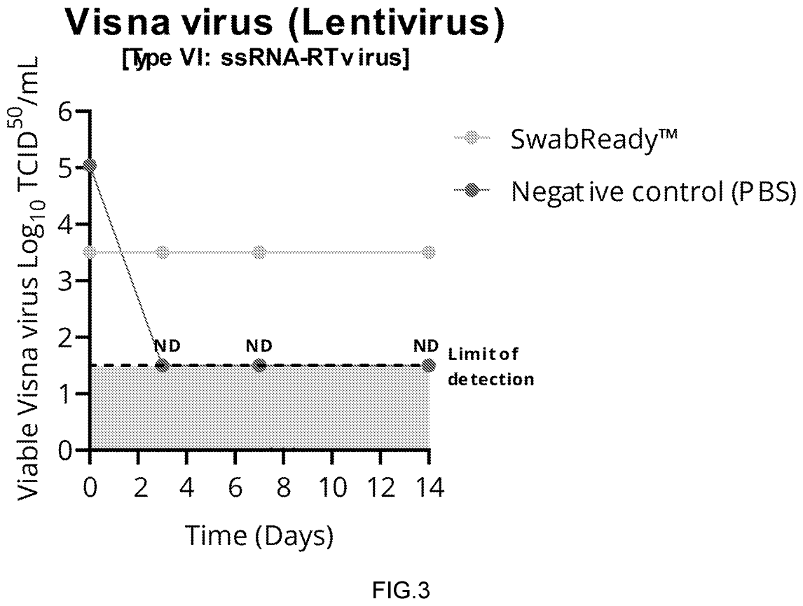

After 0 days incubation, an average of 5.04 Log 10TCID₅₀ mL-1 viable Visna virus was recovered from the PBS control. Following incubation of Visna virus in PBS for 3, 7 and 14 days no viable virus was observed, therefore the recovery of Visna virus in Atelerix SwabReady™ at all time points was compared to the Day 0 PBS control. Following 3, 7 and 14 day incubation, an average of 3.50 Log 10TCID₅₀ mL-1 viable Visna virus was recovered from Atelerix SwabReady™, demonstrating that Atelerix SwabReady™ was able to sustain detectable levels of viable Visna virus for at least 14 days (Table 4, FIG. 3).

TABLE 4
Average Log10TCID₅₀ mL−1 recovery for Visna virus following
incubation in Atelerix SwabReady ™ for up to 14 days,
when compared to the Phosphate buffered saline control at Day 0.

| | Average viable Visna virus | | | |
|---|---|---|---|---|
| | (Log₁₀TCID₅₀ mL⁻¹) | | | |
| Test agent | Day 0 | Day 3 | Day 7 | Day 14 |
| Phosphate buffered | 5.04 | N/A* | N/A* | N/A* |
| saline control | | | | |
| Atelerix SwabReady ™ | 3.50 | 3.50 | 3.50 | 3.50 |

*No viable virus remaining in phosphate buffered saline control after Day 0.

### DISCUSSION

Atelerix SwabReady™ Hydrogel was assessed for its viral preservation efficacy over 14 days in comparison with Virocult® viral transport medium. The viability of human coronavirus 229E was assessed at 5 time points.

Atelerix SwabReady™ demonstrated enhanced ability to preserve the viability of human coronavirus for up to 14 days when compared to Virocult® viral transport medium. Preserving viral viability over time presents a significant challenge to large scale viral testing and these results show Atelerix SwabReady™ was able to sustain detectable levels of viable human coronavirus 229E stably for at least 14 days. Viral detection was determined using the TCI D50 titration method, which has a minimum level of detection of 1.50 Log 10 TCID₅₀/mL and quantifies the complete infectious virions present in a solution.

Raw Data

TABLE 5
Log TCID50/mL for human coronavirus 229E (CoV 229E) following
incubation in Atelerix SwabReady ™ for up to 14 days.
Atelerix SwabReady ™ CoV 229E Log TCID₅₀/mL

| Replicate | Day 3 | Day 5 | Day 7 | Day 10 | Day 14 |
|---|---|---|---|---|---|
| N = 1 | 3.67 | 2.50 | 2.50 | 2.50 | 2.50 |
| N = 2 | 3.50 | 2.50 | 2.50 | 2.50 | 2.50 |
| N = 3 | 3.83 | 2.50 | 2.50 | 2.50 | 2.50 |
| Average | 3.67 | 2.50 | 2.50 | 2.50 | 2.50 |

TABLE 6
Log TCID50/mL for human coronavirus 229E (CoV 229E) following
incubation in Virocult ® viral transport medium for up to 14 days.
Virocult ® VTM CoV 229E Log TCID₅₀/mL

| Replicate | Day 3 | Day 5 | Day 7 | Day 10 | Day 14 |
|---|---|---|---|---|---|
| Rep 1 | 1.83 | 1.50 | 1.67 | 1.50 | 1.50 |
| Rep 2 | 2.50 | 1.50 | 1.50 | 1.50 | 1.50 |
| Rep 3 | 2.50 | 1.50 | 1.83 | 1.50 | 1.50 |
| Average | 2.28 | 1.50 | 1.67 | 1.50 | 1.50 |

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. A reversibly cross-linked hydrogel comprising a sample, wherein the sample comprises extracellular nucleic acid.

2. The hydrogel of claim 1, wherein the extracellular nucleic acid is within a lipid capsule.

3. The hydrogel of claim 1, wherein the extracellular nucleic acid is in a viral vector or a viral particle.

4. (canceled)

5. The hydrogel of claim 1, wherein the sample further comprises an aqueous buffer, optionally wherein the aqueous buffer is a salt-based buffer.

6. (canceled)

7. The hydrogel of claim 1, wherein the hydrogel comprises cross-linked alginate, optionally wherein the hydrogel comprises cross-linked calcium-alginate, strontium-alginate, barium-alginate, magnesium-alginate and/or sodium-alginate.

8. The hydrogel of claim 7, wherein the cross-linked alginate comprises from about (w/v) to about 5.0% (w/v) calcium alginate.

9. The hydrogel of claim 1, wherein the extracellular nucleic acid is RNA or DNA.

10. The hydrogel of claim 1, wherein the hydrogel is packaged in a sealed receptacle, optionally wherein the sealed receptacle is a vial, tube, flask, dish, vessel, or plate.

11. (canceled)

12. A method of preparing a sample comprising extracellular nucleic acid for storage and/or transportation from a first location to a second location, the method comprising the steps of:
i) contacting the sample with a hydrogel-forming polymer; and
ii) polymerising the polymer to form a reversibly cross-linked nucleic acid-containing hydrogel.

13. The method of claim 12, further comprising mixing the extracellular nucleic acid with an aqueous buffer prior to step (i), optionally wherein the aqueous buffer is a salt-based buffer.

14. The method of claim 12, wherein the method comprises sealing the nucleic acid-containing hydrogel into a receptacle for storage or transportation from the first location to the second location, optionally wherein the sealed receptacle is a vial, tube, flask, dish, vessel, or plate; and optionally wherein the method further comprises iii) dispatching the sealed receptacle for transportation from the first location to the second location.

15. (canceled)

16. A method of transporting a sample comprising an extracellular nucleic acid from a first location to a second location, the method comprising the steps of:
(a) obtaining a reversibly cross-linked nucleic acid-containing hydrogel generated by i) contacting the sample with a hydrogel-forming polymer; and ii) polymerising the polymer to form a reversibly cross-linked nucleic acid-containing hydrogel; or obtaining a reversibly cross-linked nucleic acid-containing hydrogel according to claim 1;
(b) transporting the hydrogel from the first location to the second location; and
(c) optionally releasing the nucleic acid from the hydrogel at the second location.

17. (canceled)

18. A method of storing a sample comprising an extracellular nucleic acid, the method comprising the steps of:
(a) obtaining a reversibly cross-linked nucleic acid-containing hydrogel generated by i) contacting the sample with a hydrogel-forming polymer; and ii) polymerising the polymer to form a reversibly cross-linked nucleic acid-containing hydrogel; or obtaining a reversibly cross-linked nucleic acid-containing hydrogel according to claim 1;
(b) storing the hydrogel; and
(c) optionally releasing the nucleic acid from the hydrogel after storage.

19. (canceled)

20. A method for fulfilling an order or request for an extracellular nucleic acid, the method comprising the steps of:
a) receiving an order or request for an extracellular nucleic acid;
b) obtaining a reversibly cross-linked nucleic acid-containing hydrogel generated by i) contacting the sample with a hydrogel-forming polymer; and ii) polymerising the polymer to form a reversibly cross-linked nucleic acid-containing hydrogel; or obtaining a reversibly cross-linked nucleic acid-containing hydrogel according to claim 1; and
c) dispatching the sample for transportation; or transporting the sample to the location specified in the order or request.

21. The method of claim 12, wherein the extracellular nucleic acid is within a lipid capsule.

22. The method of claim 12, wherein the extracellular nucleic acid is in a viral vector or a viral particle.

23. (canceled)

24. The method of claim 12, wherein the sample further comprises an aqueous buffer, optionally wherein the aqueous buffer is a salt-based buffer.

25. (canceled)

26. The method of claim 12, wherein the hydrogel comprises cross-linked alginate, optionally wherein the hydrogel comprises cross-linked calcium-alginate, strontium-alginate, barium-alginate, magnesium-alginate and/or sodium-alginate.

27. The method of claim 26, wherein the cross-linked alginate comprises from about (w/v) to about 5.0% (w/v) calcium alginate.

28. The method of claim 12, wherein the extracellular nucleic acid is RNA or DNA.

29. (canceled)
